(19) United States
(12) Patent Application Publication
(10) Pub. No.: US 2025/0382662 A1
(43) Pub. Date: Dec. 18, 2025

(54) PROTEIN LINKER SYSTEM FOR CONJUGATION AND RELEASE OF OLIGONUCLEOTIDES

(71) Applicant: Celldom, Inc., San Diego, CA (US)

(72) Inventors: Eugene TKACHENKO, Del Mar, CA (US); Milos BaBIC, San Diego, CA (US)

(73) Assignee: Celldom, Inc., San Diego, CA (US)

(21) Appl. No.: 19/087,485

(22) Filed: Mar. 22, 2025

Related U.S. Application Data
(60) Provisional application No. 63/569,134, filed on Mar. 23, 2024.
(60) Provisional application No. 63/676,258, filed on Jul. 26, 2024.

(51) Int. Cl. C12Q 1/6823

(52) U.S. Cl. CPC C12Q 1/6823

(57) ABSTRACT

The invention provides a protein linker system for conjugating oligonucleotides to the surface of a substrate. Subjecting the protein linker system to a protease releases conjugated oligonucleotides from the surface of the substrate. The inventive protein linker system avoids the use of chemical-cleavable and photo-cleavable oligonucleotide linkers, and permits the use of plastic substrates which maintain their shape and size during changes in salt and detergent concentrations in the substrate environment.

### CROSS-REFERENCE TO RELATED APPLICATIONS

This application claims the benefit of provisional application No. 63/676,258, filed Jul. 26, 2024, and provisional application No. 63/569,134, filed Mar. 23, 2024. The entire contents of these applications are incorporated herein by reference for all purposes.

### FIELD OF THE INVENTION

The invention generally relates to biologics. More particularly, the invention relates reagents, kits, devices and methods for use with oligonucleotides.

### BACKGROUND OF THE INVENTION

Oligonucleotides are commonly bound to beads by direct conjugation to a reactive group on the surface of the beads. To increase accessibility of enzymes and nucleic acids to the bound oligonucleotides, hydrophilic linkers, such as PEG, are used. PEG and other hydrophilic molecules also help to prevent the enzyme denaturation properties, and other negative effects, of hydrophobic bead surfaces. To increase the number of oligonucleotides attached on the bead, branched molecules such as multi-arm PEGs are used. Release of the oligonucleotides, or nucleic acid products created by enzymatic reaction(s) (e.g., ligation, transcription and reverse transcription reactions) on the attached oligonucleotides, can be done by cleaving the oligonucleotides with enzymes (e.g., USER), by employing a cleavable chemical linker, or by dissolving the bead if the bead is made of hydrogel.

Consistent size is important for bead sealing assays wherein beads form a seal at the openings of microwells in an array. Thus, the size of the beads in bead sealing assays should not change due to buffer exchanges which introduce variations in salt and detergent concentrations. The need for consistent bead sizes in bead sealing assays eliminates the use of hydrogel beads as hydrogel beads shrink when salt concentrations are increased. Plastic beads are well suited for bead sealing assays because of their resistance to changes in size due to buffer exchanges, commercial availability of monodisperse beads with various porosities, and high-density functional coupling groups.

However, plastic beads have hydrophobic surfaces which are difficult to block with commonly used hydrophilic oligonucleotide linkers, such as PEG. Another problem with plastic beads is nucleic acid release. This release often requires modifications to the nucleic acid to make it amenable to specific enzymes (e.g. USER), which can be slow and have low release efficiency. The use of conventional chemical oligonucleotide release systems with plastic beads is expensive and care must be taken to avoid the release chemicals from contacting buffers during buffer exchanges. Photocleavable linkers provide another option, though photocleavable linkers are expensive and tend to be unstable requiring a bead wash before the beads can be applied in an assay.

What is needed in the art therefore is an efficient, stable and inexpensive linking system that permits oligonucleotides to be attached to plastic beads and other hydrophobic substrates while coating their hydrophobic surfaces. An oligonucleotide linking system that avoids washing steps and the exposure of release chemicals to reaction mixtures due to bead shrinkage are also needs in the current state of the art.

### SUMMARY OF THE INVENTION

The invention provides reagents, devices, kits and methods for a protein-based linking system that attaches oligonucleotides to the surface of beads and other substrates. The protein-based linker system allows enzymatic reactions to be conducted on attached oligonucleotides, including, without limitation, ligation and transcription reactions. The reagents, devices, kits and methods of the invention also provide a protease-based release mechanism for oligonucleotides immobilized on the surface of beads and other substrates.

The inventors surprisingly discovered that the introduction of a protein layer between the solid surface of a hydrophobic substrate and oligonucleotides attached thereto resolves the problem of enzyme incompatibility by creating a physiological hydrogel layer. Without being bound by any particular theory or mechanism, the protein layer also multiplies the number of oligonucleotide binding sites on hydrophobic substrates by utilizing the primary amines, carboxyls and thiols of the proteins on the layer, and creates an easy way and efficient way to remove substrate-immobilized nucleic acids by a simple protease reaction.

### BRIEF DESCRIPTION OF THE DRAWINGS

FIG. 1 shows the results of amplification of oligonucleotides obtained directly from the surface of a bead substrate.

FIG. 2 shows the results of amplification of oligonucleotides released from the surface of a bead substrate using the protein linker system of the invention.

### DETAILED DESCRIPTION

The invention provides reagents, devices, kits and methods that incorporate a protein linker system for releasably conjugating oligonucleotides to a substrate. The protein linker system links oligonucleotides to beads and other substrates by a protein conjugation that is cleavable by enzymatic reaction. Thus, the inventive protein linker system provides an efficient, innocuous means for releasing oligonucleotides from substrates and avoids the deficiencies associated with releasing oligonucleotides from substrates using known substrate-oligonucleotide conjugation means.

In addition to providing an efficient, innocuous means for releasing oligonucleotides from a substrate, and without being limited to any particular theory or mechanism, the proteins in the inventive linker system form a protective hydrogel coating on substrates that prevents the surface of substrates from reacting with the reagents and biological molecules used and analyzed in assay reactions. Moreover, the protein linker system increases the number of oligonucleotide binding sites as oligonucleotides are permitted to bind the primary amine, carboxyl and thiol groups of the linker protein which forms a coating on the surface of substrates incorporating the inventive system.

In some non-limiting embodiments, the invention provides a method of releasably linking an oligonucleotide to a substrate by a cleavable protein linker. The method can be practiced by providing a substrate, contacting the substrate with at least one linker protein under suitable reaction conditions to permit the linker protein to conjugate to the surface of the substrate thereby coating at least a portion of the surface of the substrate with the linker protein. The linker protein can conjugate to the surface of the substrate by hydrophobicity in instances where the surface of the substrate and at least a portion of the linker protein contains hydrophobic domains. As used herein, the term “conjugate,” “conjugated,” “conjugates,” “conjugation,” and the like refer to the joining of one substance to another substance, including, without limitation, by means of hydrophobic-hydrophobic interaction, hydrogen bonding, ionic bonding, covalent bonding, induced dipole, or combinations thereof.

In some embodiments, the surface of the substrate is functionalized for bioconjugation to the linker protein. The surface of the substrate can be functionalized with cross-linking agents or other modifications. The surface of the substrate can be functionalized with agents to bind the carboxyl, thiol, primary amine and/or carbonyl groups of the linker protein. Non-limiting examples of cross-linking agents for use with the invention include, but are not necessarily limited to, isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, fluorophenyl esters, and combinations thereof. Cross-linking agents for use with the invention can further include the agents disclosed in the Thermo Scientific® Crosslinking Technical Handbook (Copyright 2012), the entire contents of which are incorporated herein by reference for all purposes.

With the linker protein conjugated to the surface of the substrate, the linker protein is contacted with at least one oligonucleotide under reaction conditions sufficient to permit the linker protein to conjugate the oligonucleotide. The linker protein can be conjugated to the oligonucleotide through interaction of at least one of the amine (e.g., primary amine), carboxyl and thiols groups of the linker protein forming a coating on the surface of the substrate. In some embodiments, the linker protein is pegylated before or after it is conjugated to surface of the substrate. The linker protein can be pegylated with high molecular weight polyethylene glycol. The linker protein can be pegylated with PEG-linkers having an amine-reactive group, such as an ester for example. Pegylation of the linker protein can increase the number of oligonucleotide binding sites on the surface of the substrate and make the surface of the substrate more hydrophilic. The linker protein can be modified to include exogenous carboxyl, thiol, and amine groups to facilitate conjugation to the oligonucleotide.

In some embodiments of the invention, the linker protein is modified with one or more click chemistry reagents to facilitate the linker protein's conjugation of the oligonucleotide. The linker protein can be so modified before or after the linker protein is conjugated to the surface of the substrate. The linker protein can be modified by click chemistry reagents, wherein the primary amines of the linker protein are modified to facilitate binding of the oligonucleotide. The linker protein can be modified with one or more click chemistry reagents that include, but are not necessarily limited to, one or more of a thiol, maleimide, azide, DBCO, BCN, alkyne, TCO (trans-cyclooctene), and tetrazine.

It will be appreciated that the steps of the inventive method can have one or more intervening wash steps. For example, the substrate can be washed after linker protein is conjugated to the surface of the substrate to remove protein that fails to conjugate to the surface of the substrate.

Linker proteins for use with the invention can be any protease labile protein suitable for conjugating an oligonucleotide to the substrate, or otherwise capable of being modified to conjugate an oligonucleotide as disclosed herein. In some non-limiting embodiments, the linker protein is at least one of albumin and casein. In a preferred embodiment, the linker protein is albumin. Without being bound by any particular theory or mechanism, the use of the linker protein coats the surface of the substrate and prevents the surface from engaging in non-specific reactions with biological materials, such as oligonucleotides and enzymes, while simultaneously amplifying the number of binding sites on the substrate.

In some embodiments, the invention provides a method of manipulating an oligonucleotide. The method can be practiced by manipulating oligonucleotides after they are conjugated to the linker protein. The manipulation can be carried out while oligonucleotides are conjugated to the linker protein, or after the oligonucleotides are released from the linker protein by use of a protease. Such manipulations can include, but are not necessarily limited to, extension, ligation to incorporate a desired domain, assay analysis, amplification, or a combination thereof. Ligation of the desired domain can be ligation of the one or more oligonucleotides to a primer site, an oligonucleotide barcode (e.g., a DNA barcode), an oligonucleotide probe (e.g, a DNA probe), a capture motif, or combinations thereof. Extension of the one or more conjugated oligonucleotides can be accomplished by cDNA extension, such as in a reverse transcriptase reaction, for example. The oligonucleotides can be DNA, RNA, or a combination thereof. The manipulations disclosed herein can be carried out in the performance of a single-cell expression analysis.

In the methods disclosed herein, oligonucleotides conjugated to the linker protein on the surface of the substrate (i.e., the linker protein-oligonucleotide complex) can be efficiently and stably released from the substrate by contacting the linker protein-oligonucleotide complex with a protease. The protease used to release oligonucleotides from the linker protein-oligonucleotide complex can be a non-specific protease. One non-limiting example of a protease suitable for use with the invention includes Proteinase K. In some embodiments, the oligonucleotide can be subjected to further analysis and processes after release, such as, for example, hybridization reactions, amplification, sequence analysis, or combinations thereof. For example, the one or more oligonucleotides can be released from the linker protein-oligonucleotide complex by contacting the linker protein-oligonucleotide complex with a protease after an mRNA is reverse-transcribed.

In some embodiments, the invention provides a substate incorporating the inventive protein linker system. At least a portion of the surface of the substrate can be coated with a linker protein conjugated to the surface of the substrate. The linker protein can be conjugated to the surface of the substrate by a hydrophobic-hydrophobic interaction between the surface of the substrate and hydrophobic domains of the linker protein. In some embodiments, the surface of the substrate is functionalized with agents to bind the carboxyl, thiol, primary amine and/or carbonyl groups of the linker protein. The surface of the substrate can be functionalized with a cross-linking agent as disclosed herein. The linker protein can be any protein capable of conjugating to the surface of the substrate and conjugating to an oligonucleotide. The linker protein can be at least one of albumin and casein. In a preferred embodiment, the linker protein is albumin. In a further embodiment, the substrate is incorporated in a kit, wherein the kit includes a protease suitable for cleaving the linker protein. The protease can be a non-specific protease, such as Proteinase K, for example. The linker protein can be modified by click chemistry reagents to facilitate the conjugation of the linker protein to oligonucleotides. The linker protein can have amine groups modified by click chemistry. The linker protein can be modified by click chemistry reagents selected from one or more of thiol, maleimide, azide, DBCO, BCN, alkyne, TCO (trans-cyclooctene), and tetrazine.

In other embodiments, the invention provides a kit for conjugating a linker protein to a substrate. Such kits can comprise a substrate and one or more linker proteins as disclosed herein. The kit can further include reagents necessary and tools necessary for conjugating the linker protein to the substrate. The kit can further include one or more reagents suitable for functionalizing a surface of the substrate with one or more agents for functionalizing the substrate to bind the carboxyl, thiol, primary amine and/or carbonyl groups of the linker protein. The kit can include cross-linking agent as disclosed herein. The linker protein can be one or more of albumin and casein. The kit can further include at least one protease suitable for cleaving the linker protein. The protease can be a non-specific protease, such as, for example, Proteinase K.

In yet other embodiments, the invention provides a kit for conducting an oligonucleotide assay or procedure. Such a kit can include a kit for coupling a linker protein to a substrate as disclosed herein, and further include reagents suitable for conducting an oligonucleotide assay or procedure. The reagents can include reagents suitable for conducting one or more of oligonucleotide extension, ligation of an oligonucleotide to incorporate a desired domain, oligonucleotide amplification (e.g., PCR), transcription, reverse transcription, or combinations thereof.

Kits according to the invention can be assembled in packaging with instructions for conducting a desired protocol, procedure or experiment, including, without limitation, linking and releasing an oligonucleotide from a substrate by a protease reaction as disclosed herein.

The inventive kits, devices and methods can be practiced with any substrate suitable for conjugating a linker protein to the surface of the substrate as disclosed herein. The substrate can be any substrate wherein linking of oligonucleotides to a substrate is desired. The substrate can be a bead, slide, dish, cover slip, culture plate, culture bottle, microwell array, vile, test tube, filter, or combinations thereof. In some preferred embodiments, the substrate is a bead. In some preferred embodiments, the substrate is plastic. The substrate can be hydrophobic. The substrate can be a hydrophobic plastic, for example.

Non-limiting examples of substrate materials for use with the invention include, but are not necessarily limited to, polymethylmethacrylate, epoxy, silicon, fused-silica, glass, a polymer, a metal, alumina, an elastomer, polydimethylsiloxane, agarose, and a hydrogel, or any combination thereof. Examples of beads suitable for use with the invention include, but are not limited to, agarose beads, plastic beads, magnetic beads, glass beads, Dynabeads®, MACS® microbeads, silica beads, silica-like beads, and BcMag™ Carboxy-Terminated Magnetic Beads. The beads can be polymethylmethacrylate microspheres. The beads can have a density of between about 1 g/cm³ and about 4 g/cm³. The beads can have a density of up to 2 g/cm³, up to 3 g/cm³, up to 4 g/cm³, or up to any density that intervenes the specifically listed densities. The beads can have a density of at least 1 g/cm³, at least 2 g/cm³, at least 3 g/cm³, at least 4 g/cm³, or at least any density that intervenes these specifically listed densities. The beads can be associated with (e.g., impregnated with) quantum dots or fluorescent dyes to make them fluorescent in one fluorescence optical channel or multiple optical channels. The beads can be magnetic. The beads can be associated with iron oxide or chromium oxide to make them paramagnetic or ferromagnetic.

The inventive linker system can be used to link and release one or more oligonucleotides from a substrate. The oligonucleotides can be primers, DNA barcodes, DNA probes, cDNA, mRNA, capture motifs, or combinations thereof. In some embodiments, the protein linker system conjugates oligonucleotides to beads for use in microwell-based assays, including, but not limited to, single cell expression assays. The beads incorporating the inventive protein linker system can be used in a sealed microwell assay, including single cell analysis, such as disclosed in U.S. Pat. No. 11,969,702, the entire contents of which are incorporated herein by reference for all purposes.

The inventive protein linker system can be used to conjugate one or more oligonucleotides to beads for use in single cell mRNA expression analysis. In some non-limiting embodiments, the inventive protein linker system conjugates beads to oligonucleotide moieties for use in single cell expression analysis, wherein the oligonucleotide moieties comprise (i) a cell marker barcode portion that is unique to each bead within a plurality of beads (e.g., a bead library), (ii) a capture portion that is complimentary to a targeted oligonucleotide (e.g., a mRNA expression product), and optionally (iii) a capture barcode portion that is unique to each capture portion. Individual beads can be conjugated to a plurality of the oligonucleotide moieties, wherein the moieties are the same as, or different from one another. For different moieties, a bead can be conjugated to a plurality of oligonucleotide moieties having the same cell marker barcode portion, but having capture portions that are different from one another, wherein the capture portions are each identified by their own individual capture barcode portion. Suitable oligonucleotide moieties for use with the invention include, but are not limited to, those disclosed in U.S. patent application publication No. 2016/0289669, the entire contents of which are incorporated herein by reference for all purposes.

In some non-limiting single cell analysis applications, beads having an oligonucleotide moiety conjugated thereto by the inventive protein linker system are introduced to a microwell array that is loaded with cells. The beads occupy the microwells and a hybridization reaction is caried out under conditions sufficient to permit the capture portion of the moiety to hybridize with one or more targeted oligonucleotide expression products that are released from the cells by exocytosis, cell lysis, or a combination thereof. A protease is then introduced to the array to release the moieties with hybridized oligonucleotide expression products into the microwells. The protease can be introduced by, for example, contacting the upper surface of the microwells with the protease in a reaction buffer to permit the protease to occupy the microwells by diffusion. Alternatively, the protease can be introduced to the microwells by microfluidics for microwells so adapted. The beads denuded of the moieties with hybridized oligonucleotide expression products are then optionally removed from the array, and the moieties with hybridized oligonucleotide expression products are collected from the microwells and subjected to sequence analysis, extension, amplification, or a combination thereof.

It will be appreciated that the steps for single cell analysis using the protein linker system as disclosed herein need not be conducted in any particular order. For example, the beads can be introduced to the microwells having cells therein and the oligonucleotide moieties then conjugated to the beads by the protein linker system as disclosed herein, then the remaining steps of the assay conducted. Alternatively, the beads can have one or more oligonucleotide portions conjugated thereto by the inventive protein linker system, and the oligonucleotides then manipulated to include at least one of extension, ligation to incorporate a desired domain, amplification, or a combination thereof. Ligation of the desired domain can be ligation of the linked oligonucleotide to a primer site, a DNA barcode, a DNA probe, a capture motif, or combinations thereof. Extension of the conjugated oligonucleotides can be accomplished by cDNA extension, such as in a reverse transcriptase reaction, for example. In still other embodiments, beads bearing oligonucleotide moieties can be used to carry out hybridization of the capture portion of the moieties to a targeted mRNA expression product, and the reverse transcription of the targeted mRNA conducted, followed by the release of the oligonucleotide moieties from the beads by the introduction of a protease.

Non-limiting methods for carrying out the amplification and correlation of mRNA expression products with the cell marker barcode portions and capture barcode portions of the oligonucleotide moieties are disclosed in the following references, the entire contents of which are incorporated herein by reference in their entirety for all purposes: U.S. patent application publication No. 2016/0289669; Bose et al. Genome Biology (2015) 16:120; and Fan et al. Science (2015) Vol 347, Issue 6222, P. 1258367-1.

### EXAMPLE

Beads were conjugated to albumin, and poly-T oligonucleotides were associated with the albumin matrix. Single beads were associated with individual cells, which were then lysed to capture mRNAs on the bead via their poly-A tails. Reverse transcription was then performed to generate cDNAs. Beads were thoroughly washed. A negative control was processed in the same way, except that beads were never associated with cells.

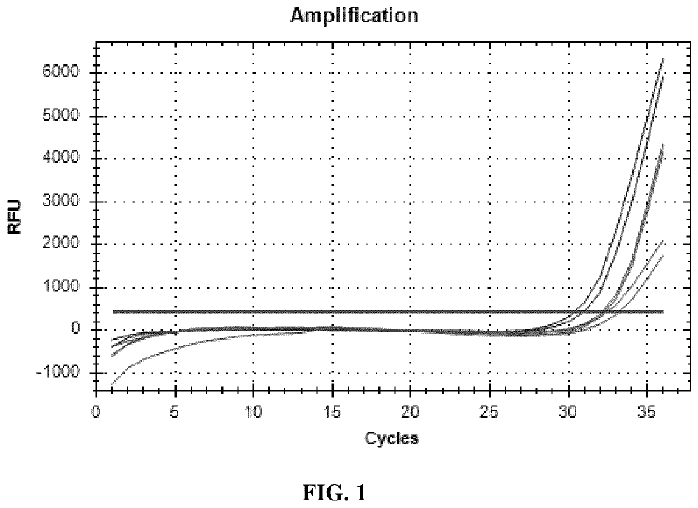

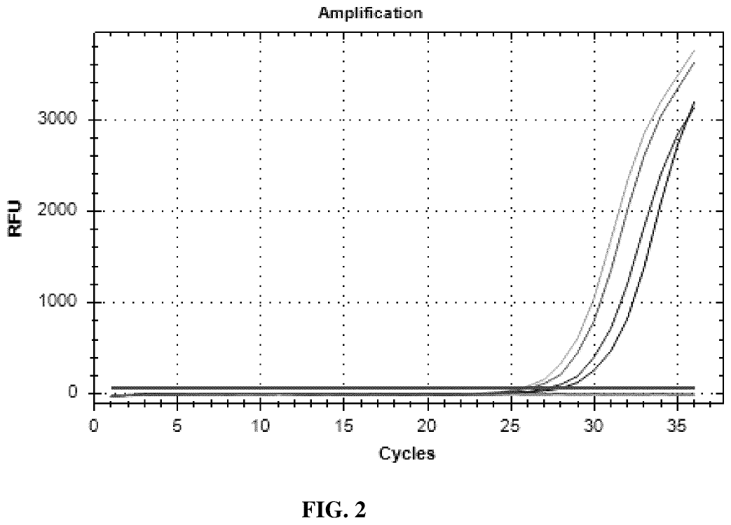

Pairs of primers specific for two housekeeping genes (GAPDH and HPRT) can be used to amplify specific cDNAs, showing their presence on the bead. In one experiment (FIG. 1) targets were amplified directly from the bead (red trace shows the negative control). In the second experiment (FIG. 2), beads were treated with thermolabile proteinase K, which was then inactivated; the supernatant of the reaction was used as the template for PCR amplification.

While the qPCR traces clearly show the presence of cDNAs in both experiments, the signal is lower for the on-bead amplification (left), and the negative control beads (red trace) also show a false signal due to nonspecific amplification of oligonucleotides on the bead surface.

In comparison, cDNAs released from the bead show a stronger and cleaner signal, without any amplification from the negative control samples.

### Efficiency of Release

First, we attached albumin to the beads. Second, we attached an initial oligonucleotide “handle” (SEQ ID NO:1) to immobilized albumin. Then, a complementary oligonucleotide was added to form the basic hybridization/ligation platform (SEQ ID NO:2). The final structure was as follows:

(bead)-albumin-PEG-TCAGAGGGTCTATACGTTACATAGUGACUGGA-3′
3′-AGTCTCCCAGATATGCAAUGTATCACTGACCTCAAGTCTGCACACGAGAAG-5′

Note the uracil residues that are bolded within the sequence.

We then ligated a 27 bp oligo (SEQ ID NO:3) with attached fluorophore (fluorophore bolded):

5′-GTTCAGACGTGTGCTCTTCCGATCTTT/Cy3/-3′

For this oligo, the resulting structure is:

(SEQ ID NO: 4)
(bead)-albumin-PEG-5′
TCAGAGGGTCTATACGTTACATAGUGACUGGAGTTCAGACGTGTGCTCT
TCCGATCTTT/Cy3-3′
(SEQ ID NO: 5)
3′-AGTCTCCCAGATATGCAAUGTATCACTGACCTCAAGTCTGCACACG
AGAAG-5′

We then used a fluorimeter (DeNovix DS-11 FX) to determine the fluorescence of beads before and after release using the standard method (USER enzyme) vs. proteinase release. As oligonucleotides are released, the bead fluorescence (shown in relative fluorescence units, RFU) is reduced, leading to results that are closer to non-fluorescent beads.

| | RFU |
|---|---|
| non-fluorescent beads | 131 |
| starting fluorescent beads | 3,928 |
| after 1 h incubation at 37° C. with standard USER enzyme | 3,655 |
| after overnight incubation at 45° C. with | 612 |
| temperature-stable USER enzyme | |
| after 10 minute protease digestion at 37° C. | 337 |

Release of oligonucleotides with USER is very inefficient, requiring long incubation times and higher temperatures. In comparison, release with proteinase K is >90% complete after 10 minutes.

### REFERENCE TO AN ELECTRONIC SEQUENCE LISTING

The contents of the electronic sequence listing (Name of File: AppNo19084485S.xml; Size: 9,743 bytes; and Date of Creation: Sep. 2, 2025) is herein incorporated by reference in its entirety.

1. A method of manipulating an oligonucleotide, comprising:
a) providing a substrate;
b) contacting a surface of the substrate with at least one linker protein under conditions sufficient to conjugate the at least one linker protein to the surface of the substrate thereby coating at least a portion of the surface of the substrate with the at least one linker protein; and
c) contacting the at least one linker protein conjugated to the surface of the substate with at least one oligonucleotide under conditions sufficient to conjugate the at least one linker protein to the at least one oligonucleotide thereby providing at least one linker protein-oligonucleotide complex.

2. The method of claim 1, wherein (i) the surface of the substrate is hydrophobic, (ii) at least a portion of the at least one linker protein is hydrophobic, and (iii) the at least one linker protein is conjugated to the surface of the substrate by hydrophobicity.

3. The method of claim 1, wherein the surface of the substrate is functionalized to conjugate at least one of carboxyl, thiol, primary amine and carbonyl groups of the at least one linker protein.

4. The method of claim 1, wherein the at least one linker protein is selected from albumin, casein and a combination thereof.

5. The method of claim 1, further comprising subjecting the at least one oligonucleotide of the at least one linker protein-oligonucleotide complex to a manipulation.

6. The method of claim 5, wherein the manipulation includes at least one of extension, ligation, amplification, hybridization, and sequence analysis.

7. The method of claim 1, further comprising contacting the at least one linker protein-oligonucleotide complex with a protease thereby cleaving the at least one protein of the at least one linker protein-oligonucleotide complex and releasing the at least one oligonucleotide from the surface of the substrate.

8. The method of claim 7, wherein the protease is Proteinase K.

9. The method of claim 7, further comprising subjecting the released at least one oligonucleotide to a manipulation.

10. The method of claim 9, wherein the manipulation includes at least one of extension, ligation, amplification, hybridization, and sequence analysis.

11. The method of claim 1, wherein the at least one linker protein includes an amine group and the at least one oligonucleotide is conjugated to the at least one linker protein by the amine group.

12. The method of claim 1, wherein the at least one linker protein is modified by one or more click chemistry agents and the at least one oligonucleotide is conjugated to the at least one linker protein by the one or more click chemistry reagents.

13. The method of claim 12, wherein the one or more click chemistry agents is selected from thiol, maleimide, azide, DBCO, BCN, alkyne, TCO (trans-cyclooctene), tetrazine, and combinations thereof.

14. The method of claim 1, wherein the surface of the substrate is hydrophobic.

15. The method of claim 1, wherein the substrate is made of a plastic.

16. The method of claim 1, wherein the substrate is a bead.

17. The method of claim 1, wherein the at least one linker protein is pegylated.

18. The method of claim 17, wherein the at least one linker protein is pegylated before or after the at least one linker protein is conjugated to the at least one oligonucleotide.

19. The method of claim 1, wherein the at least one linker protein is selected from albumin and casein.


SEQUENCE LISTING (5 sequences)

SEQ ID NO: 1 - DNA, 32 bp, synthetic construct
  source 1..32: mol_type = other DNA
  modified_base 25: mod_base = OTHER; mod_base = OTHER; note = thymine at location 25 is uracil
  modified_base 29: mod_base = OTHER; mod_base = OTHER; note = thymine at location 29 is uracil
tcagagggtctatacgttacatagtgactgga

SEQ ID NO: 2 - DNA, 51 bp, synthetic construct
  source 1..51: mol_type = other DNA
  modified_base 19: mod_base = OTHER; note = thymine at location 19 is uracil
agtctcccagatatgcaatgtatcactgacctcaagtctgcacacgagaag

SEQ ID NO: 3 - DNA, 28 bp, synthetic construct
  source 1..28: mol_type = other DNA
gttcagacgtgtgctcttccgatctttc

SEQ ID NO: 4 - DNA, 60 bp, synthetic construct
  source 1..60: mol_type = other DNA
  modified_base 25: mod_base = OTHER; mod_base = OTHER; note = thymine at location 25 is uracil
  modified_base 29: mod_base = OTHER; mod_base = OTHER; note = thymine at location 29 is uracil
tcagagggtctatacgttacatagtgactggagttcagacgtgtgctcttccgatctttc

SEQ ID NO: 5 - DNA, 51 bp, synthetic construct; identical to SEQ ID NO: 2
  source 1..51: mol_type = other DNA
  modified_base 19: mod_base = OTHER; mod_base = OTHER; note = thymine at location 19 is uracil